(19) United States
(12) Patent Application Publication
(10) Pub. No.: US 2012/0217201 A1
(43) Pub. Date: Aug. 30, 2012

(54) METHOD FOR ENHANCING BIOLOGICAL WATER TREATMENT

(75) Inventors: Huu Hao Ngo, New South Wales (AU); Wenshan Guo, New South Wales (AU)

(73) Assignee: UNIVERSITY OF TECHNOLOGY, SYDNEY, New South Wales (AU)

(21) Appl. No.: 13/500,568

(22) PCT Filed: Oct. 6, 2010
(86) PCT No.: PCT/AU2010/001304

§ 371 Date: Apr. 5, 2012

(30) Foreign Application Priority Data
Oct. 6, 2009 (AU) 2009904857

(51) Int. Cl. C02F 9/14; C12N 1/16; C12N 1/00; C02F 1/56

(52) U.S. Cl. 210/610; 210/198.1; 210/151; 210/727; 210/728; 435/255.1; 435/243

(57) ABSTRACT

A mixture for treating water in a biological water treatment process is provided comprising an organic-based flocculant, a micronutrient; and a polymer. The flocculant, the micronutrient and the polymer are mixed in a predetermined ratio to enhance said biological water treatment. The mixture is particularly suited to enhancing flocculation of particles, reducing fouling on a surface of a membrane in a membrane bioreactor, improving uptake of phosphorous and/or nitrogen by a biomass in a biological water treatment system, improving bioactivity of a biomass and/or floc in a biological water treatment system and improving flux in a membrane bioreactor.

### FIELD OF THE INVENTION

The present invention relates to biological water and wastewater treatment particularly but not only with biological treatment systems such as membrane bioreactors. However, it will be appreciated that the invention is not limited to this particular field of use.

The term “water” used throughout this document includes any water subject or in need of treatment particularly but not only industrial and municipal wastewater, grey water, black water, domestic and agricultural effluent and the like.

### BACKGROUND OF THE INVENTION

Any discussion of the prior art throughout the specification should in no way be considered as an admission that such prior art is widely known or forms part of the common general knowledge in the field.

Biological treatment is a well-known technology in the water treatment industry. Bacteria and other microorganisms are used to remove contaminants in water by assimilating them. For example, activated sludge is a commonly used process in biological water treatment. Air is passed into water to develop a biological flocculant which acts to reduce the organic content of the waste. Activated sludge is a common method of removing pollutants from water. This process is particularly used in the treatment of domestic waste (sewage).

In an activated sludge process, large quantities of air are bubbled through wastewater containing dissolved organic substances in open aeration tanks. Oxygen is required by bacteria and other types of microorganisms present in the system to live, grow, and multiply so as to consume dissolved organic “food”, or pollutants in the waste. When the waste has been sufficiently treated, excess waste (mixed liquor) is discharged into settling tanks. The supernatant is thereby run off to undergo further treatment before discharge. The settled material, the sludge, may be returned to the head of the aeration system to re-seed the new waste entering the tank. The remaining sludge is further treated prior to disposal.

The term “activated sludge” refers to biomass produced in raw water or settled water (flocculated water after removal of settleable flocs) by the growth of organisms in aeration tanks in the presence of dissolved oxygen. Activated sludge is different from primary sludge in that the sludge contains many living organisms which can feed on the incoming water. An activated sludge process uses a mixture of aerated waste and activated sludge. The activated sludge is subsequently separated from the treated waste by settlement and may be re-used.

In biological treatment, activated sludge functions as a biological flocculant. It is believed that the biomass in the sludge secretes chemicals which assist in causing the small particles in the water to coagulate (stick together), and become heavier thereby settling out of the liquid. In water treatment operations, the process of flocculation is employed to separate suspended solids from water. Flocculation is the action of polymer compounds forming links between particles and binding the particles into large agglomerates. Segments of the polymer chain adsorb on different particles and assist in particle aggregation. Once suspended particles are flocculated into larger particles, they can usually be removed from the liquid by sedimentation, provided that a sufficient density difference exists between the suspended matter and the liquid. Such particles can also be removed or separated by media filtration, straining or floatation. The flocculation reaction not only increases the size of the particles to settle them faster, but also affects the physical nature of the flocculant, making these particles less gelatinous and thereby easier to dewater.

Flocculating agents are generally categorized into inorganic flocculants, organic synthetic polymer flocculants and naturally occurring bio-polymer flocculants. Organic synthetic polymer flocculants have been used together with inorganic flocculants because of low cost, easy handling and high efficiency. However, these flocculants can give rise to environmental and health risks during degradation. Further, polymeric flocculants do not biodegrade, which is another significant drawback relating to their use. Hence, biodegradable, naturally occurring flocculants having lower ecological impact are preferred. This is particularly the case in applications such as water reclamation and reuse.

However, naturally occurring bio-polymer flocculants are less sought-after in water treatment applications because of their lower flocculating abilities. These include low charge density, lower molecular weight and higher susceptibility to biological degradation.

A flocculant composition is disclosed in U.S. Pat. No. 6,531,531, wherein a hydrophilic polymer dispersion containing an inorganic flocculant is used to reduce the water content of the flocculant particles obtained. Another object of the invention as to reduce the water content of the sludge cake obtained after treating water. The polymer dispersion of U.S. Pat. No. 6,531,531 contains a complex mixture of acrylamide polymerised with anionic and cationic monomers, anionic salt, inorganic flocculant such as ammonium sulphate, ammonium chloride etc, non-ionic surfactant and stabiliser. While the abovementioned mixture may be effective in dewater the floc particles and the resulting sludge cake obtained, the synthetic compounds used in the mixture may still pose an environmental risk during degradation and are not biodegradable.

Effective natural flocculant products are particularly preferred in aerobic and anaerobic biological wastewater treatment processes. The biomass used in such processes is sensitive to the addition of synthetic chemicals and can often be killed by the addition of such chemicals. U.S. Pat. No. 7,048,859 discloses a method of separating biosolids from an aqueous feed stream using an organic polymer with an anionic inorganic colloid to flocculate the biosolids. However, although biological contaminants such as proteins are removed from the feed stream in this instance, the wastewater treatment process disclosed uses only flocculation with no mention of a biological treatment process. The organic polymer is also a polyacrylamide-based compound, which may be synthetic and suffer from the drawbacks disclosed above. Further, the inorganic colloid is selected from a group of compounds containing silica. Although silica occurs naturally as a trace mineral in water, silica can also present significant problems in terms of fouling of any upstream additional treatment processes.

Biological waste water treatment processes can be combined with an additional treatment step, such as membrane filtration. This combination is known as a membrane bioreactor or “MBR”. A key challenge with MBR technology is fouling of the membranes themselves. The membrane acts a physical barrier between the wastewater in the bioreactor and the treated filtrate. The micro-porous membrane allows water to pass through whilst preventing suspended solids material, micro-organisms etc from being carried into the filtrate. A great deal of research and development effort has been applied to producing membrane material that prevents or reduces fouling, and to design MBR systems that reduce the fouling effect. However, membrane fouling is still a major problem for the industry, which requires a variety of maintenance and operational interventions. This includes frequent backwashing of the membranes, chemical and physical cleaning, or intermittent pump shutdown. In addition, fouling restricts the capacity of the MBR plant by reducing the critical operating flux, which in turn means that MBR plants often operate at less than optimum capacity.

The net effect for the MBR operators is a significant cost penalty in terms of energy usage for back flushing, maintenance and materials, reduced membrane life and either underperforming plants or designed-in redundancy with increased capital expenditure.

The common strategies for fouling control include optimizing the hydrodynamic conditions in bioreactors, operating membrane systems below the critical flux, pre-treating the feed water, or conducting air scouring, membrane backwashing and cleaning. Alternative methods involve membrane coating, the addition of porous carriers for attached growth, flocculation of sludge by adding additives, and modification of the suspension by adsorption. Previously, various chemicals including synthetic or natural polymers, metal salts, resins, granular or power activated carbon have also been tested for filterability and fouling reduction in MBR mixed liquors. However, in addition to membrane fouling control, aspects of chemical addition to MBR systems such as toxicity and biodegradability and their effects on organic and nutrient removal need further investigation.

It is an object of the present invention to overcome or ameliorate at least one of the disadvantages of the prior art, or to provide a useful alternative.

### SUMMARY OF THE INVENTION

According to a first aspect, the invention provides a mixture for treating water in a biological water treatment process comprising:

an organic-based flocculant;

a micronutrient; and

a polymer;

wherein said flocculant, said micronutrient and said polymer are mixed in a predetermined ratio to enhance said biological water treatment.

The flocculant, micronutrient and polymer are preferably added to water in synergistic quantities so as to enhance said biological water treatment.

According to a second aspect, the invention provides a reagent for a biological water treatment process, said reagent comprising:

a micronutrient; and

a naturally occurring polymer;

wherein said reagent is applied to said process in synergistic quantities with a flocculant.

The reagent is preferably applied to the process prior to or simultaneously with the flocculant.

According to a third aspect, the invention provides a biological water treatment system comprising:

an organic-based flocculant;

a micronutrient; and

a polymer;

said flocculant, said micronutrient and said polymer being mixed in a predetermined synergistic ratio and added to water in a biological water treatment process.

The flocculant, micronutrient and polymer are may be mixed together in the predetermined synergistic ratio prior to addition to the water treatment process. Alternatively, the flocculant, micronutrient and polymer may be mixed in situ the water treatment process.

The biological water treatment process may include a membrane bioreactor or a submerged sponge or a submerged membrane bioreactor or any combination thereof.

According to a fourth aspect, the invention provides a biological method of treating water comprising the steps of:

adding a micronutrient, a polymer and an organic-based flocculant in a predetermined synergistic ratio to water in need of treatment; and

allowing the resultant treated water to form floc.

The biological method of treating water may further comprise removing the floc from the water. The micronutrient, polymer and organic-based flocculant may be added separately to the water. The micronutrient and/or polymer are may be added prior to or simultaneously with the flocculant. Alternatively, the micronutrient, polymer and organic-based flocculant are mixed together prior to addition to the water.

According to a fifth aspect, the invention provides a method of enhancing the efficacy of an organic-based flocculant in the biological treatment of water, said method comprising combining said flocculant with a synergistic quantity of a micronutrient and polymer either prior to or simultaneously with addition of the flocculant to the water.

Combining the micronutrient and polymer with the organic based flocculant is preferably conducted within a biological water treatment apparatus for treating the water.

According to a sixth aspect, the invention provides a method of modifying characteristics of floc produced by using a flocculant in a biological water treatment process comprising:

adding to water in need of treatment, either separately to or simultaneously with said flocculant, a micronutrient and a polymer in a predetermined synergistic ratio.

The characteristic is preferably one or more of size of the floc, biological activity, density, settling rate, viscosity, surface properties, sludge volume index (SVI) and zone settling velocity (ZSV).

According to a seventh aspect, the invention provides a method of treating water comprising the steps of:

adding to said water an organic-based flocculant, a micronutrient, and a polymer;

wherein said flocculant, said micronutrient and said polymer are mixed into said water in a predetermined synergistic ratio to enhance flocculation of particles in said water.

According to an eighth aspect, the invention provides a method of treating water comprising the steps of:

adding to said water an organic-based flocculant, a micronutrient, and a polymer;

wherein said flocculant, said micronutrient and said polymer are mixed into said water in a predetermined synergistic ratio to reduce fouling on a surface of a membrane in a membrane bioreactor used to treat said water.

According to a ninth aspect, the invention provides a method of treating water comprising the steps of:

adding to said water an organic-based flocculant, a micronutrient, and a polymer;

wherein said flocculant, said micronutrient and said polymer are mixed into said water in a predetermined synergistic ratio to improve an uptake of phosphorous and/or nitrogen by a biomass in a biological treatment system used to treat said water.

According to a tenth aspect, the invention provides a method of treating water comprising the steps of:

adding to said water an organic-based flocculant, a micronutrient, and a polymer;

wherein said flocculant, said micronutrient and said polymer are mixed into said water in a predetermined synergistic ratio to improve bioactivity of a biomass and/or floc in a biological treatment system used to treat said water.

According to an eleventh aspect, the invention provides a method of treating water comprising the steps of:

adding to said water an organic-based flocculant, a micronutrient; and a polymer;

wherein said flocculant, said micronutrient and said polymer are mixed into said water in a predetermined synergistic ratio to improve flux in a membrane bioreactor used to treat said water.

The water treatment mixture, biological water treatment system and methods of the present invention preferably comprise 20-60 parts per weight of flocculant, and the reagent of the present invention is applied with the same. This range may comprise 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 parts per weight of flocculant or any amount therebetween. The water treatment mixture, reagent, biological water treatment system and methods of the present invention preferably comprise 4-8 parts per weight of micronutrient. This range may comprise 4, 5, 6, 7, or 8 parts per weight of micronutrient or any amount therebetween. Further, the water treatment mixture, reagent, biological water treatment system and methods of the present invention preferably comprise 1-5 parts per weight of polymer. This range may comprise 1, 2, 3, 4 or 5 parts per weight of polymer or any amount therebetween.

In the water treatment mixture, biological water treatment system and methods of the present invention, flocculant is preferably provided in a quantity of between 8 and 17 mg per litre of water, and the reagent of the present invention is applied with the same. This range may comprise 8, 9, 10, 11, 12, 13, 14, 15, 16 or 17 mg per litre of water or any amount therebetween. In the water treatment mixture, reagent, biological water treatment system and methods of the present invention, micronutrient is preferably provided in a quantity of between 0.2 and 1 mg per litre of water. This range may comprise 0.2, 0.3, 0.4, 0.5, 0.6, 0.7, 0.8, 0.9 or 1.0 mg per litre of water or any amount therebetween. In the water treatment mixture, reagent, biological water treatment system and methods of the present invention, polymer is preferably provided in a quantity of between 1.5 and 3 mg per litre of water. This range may comprise 1.5, 1.6, 1.7, 1.8, 1.9, 2.0, 2.1, 2.2, 2.3, 2.4, 2.5, 2.6, 2.7, 2.8, 2.9 or 3.0 mg per litre of water or any amount therebetween.

The flocculant of the water treatment mixture, biological water treatment system and methods of the present invention is preferably biodegradable. The flocculant is more preferably a natural organic-based flocculant. In a preferred embodiment, the flocculant is a starch-based flocculant.

The micronutrient of the water treatment mixture, reagent, biological water treatment system and methods of the present invention preferably comprises a salt selected from a group consisting of iron, zinc, sodium, magnesium and manganese salts. In a preferred embodiment, the micronutrient comprises a plurality of inorganic salts. The micronutrient may comprise one or more of ferric chloride (FeCl₃), magnesium sulphate (MgSO₄), sodium sulphate (Na₂SO₄), zinc sulphate (ZnSO₄) and manganese chloride (MnCl₂). In another embodiment, the micronutrient may comprise yeast.

The polymer of the water treatment mixture, reagent, biological water treatment system and methods of the present invention preferably comprises a naturally occurring polymer. In a preferred embodiment, the polymer is chitosan.

In the reagent of the present invention, the polymer/micronutrient ratio comprises between 0.2/1.5 mg and 1/3 mg per litre of treated water. This range may comprise 0.2/1.5 mg, 0.3/1.5 mg, 0.4/1.5 mg, or 0.5/1.5 mg (1/3) per litre of treated water or any amount therebetween.

The water treatment mixture, reagent and process of the present invention at least in its preferred forms has been shown to deliver a number of key benefits in regards to use in conjunction with biological water treatment technologies. This is particularly the case with biological wastewater treatment, such as those using membrane bioreactors. It has been shown to significantly reduce organic fouling and biofouling of the membranes used in a membrane bioreactor, and also enhances the bioactivity of the biomass in the membrane bioreactor. This enhanced bioactivity leads to very high organic, phosphorous and nitrogen removal rates.

A preferred embodiment of the invention uses a naturally occurring, biodegradable, organic-based flocculant and is designed to have minimal negative impact on the natural environment, particularly a biomass in the case of a membrane bioreactor plant. The use of the water treatment mixture/reagent has been shown to improve the overall performance of a membrane bioreactor system. It is believed that the mixture/reagent contributes to reduced membrane fouling by modifying floc characteristics such as size, density, settling rates, etc, while also acting as a food source for the biomass in the MBR to enhance bioactivity of the biomass and floc.

### BRIEF DESCRIPTION OF THE DRAWINGS

The present invention will now be described by way of example only with reference to the accompanying drawings and examples in which:

FIG. 1 illustrates the effect of the addition of the water treatment mixture or reagent on the parameters of an MBR treatment system in accordance with an embodiment of the present invention;

FIG. 2 shows the results of DOC, NH4—N, T-P and T-P removal in an SMBR system using the water treatment mixture or reagent of the present invention;

FIG. 3 illustrates the comparison of DO consumption, OUR, membrane fouling and total nitrogen and phosphorus removal in MBR systems with and without the water treatment mixture or reagent of the present invention;

FIG. 4 illustrates a comparison of membrane fouling in MBR systems with and without the water treatment mixture or reagent of the present invention; and

FIG. 5 illustrates a comparison of the effluent quality of conventional SMBR and integrated SMBR using the water treatment mixture or reagent of the present invention.

### MODE (S) FOR CARRYING OUT THE INVENTION

The water treatment mixture and reagent of the present invention are designed to improve the efficacy of biological water treatment technologies. The levels of the components of the mixture of flocculant/micronutrient/polymer are selected such that they provide a synergistic effect to enhance the performance of a water treatment system. Preferred embodiments of the invention are also biodegradable and based on naturally occurring material, thus limiting the impact on the natural environment.

A particularly preferred embodiment of the mixture is designed for use in membrane bioreactors, or MBRs. The mixture is such that the impact on the biomass of an MBR plant is limited. The overall performance of MBR plants has been shown to be improved with use of the mixture of the present invention. Membrane fouling is reduced while the mixture also acts as a food source for the biomass in the MBR, thus enhancing bioactivity and improving membrane flux. The particle size distribution of floc resulting from use of the mixture of the present invention have been shown to be higher than the size distribution using conventional flocculants. High organic and phosphorus removal rates have also been achieved using the mixture/reagent of the present invention in an MBR plant.

Membrane fouling and the associated remedial actions to remove the fouling result in reduced membrane life. Application of the disclosed methods and mixtures/reagents could result in longer membrane life, and increased return on investment.

When compared to other generic and conventional flocculants and their uses, the inventive technique of the present invention demonstrates reduced membrane fouling of more than an order of magnitude improvement, whilst competing favourably against other key criteria. It also offers inherent advantages over inorganic and synthetic polymer flocculants such as being derived from a renewable source of low cost raw materials, and is easily and safely degradable in the environment after use.

The mixture of flocculant/micronutrient/polymer can be used in any aerobic and/or anaerobic biological wastewater treatment process. Demonstrating significant growth enhancing properties, the mixture is particularly suitable for use in any biological treatment process.

The water treatment mixture has been shown to offer inherent advantages over inorganic and synthetic polymer flocculants. These advantages include being derived from a renewable source of raw, low cost materials. Preferred embodiments are readily degradable in the environment after use.

Flocculants are also used extensively in the brewery, oil and paper industries and the mixture of the invention has the potential to deliver key benefits to these industries also.

Other complementary technologies have also been shown to benefit from use in conjunction with the mixture of the present invention. These include the application of a submerged sponge into the MBR. The mixture is used with a submerged sponge in an MBR system to increase biomass, reduce membrane fouling and increase nutrient removal. The submerged sponge can be combined with the mixture of the invention to simultaneously remove nitrogen and other nutrients, providing a total treatment solution with high efficiency rates.

Other studies (Ngo et al. Bioresource Technology 99 (2008) 2429-2435) have shown that a sponge-submerged membrane bioreactor system can be used for alleviating membrane fouling, enhancing permeate flux and improving phosphorous and nitrogen removals simultaneously. The sponge has been shown to be a significant attached growth media which can act as a mobile carrier for active biomass, while reducing cake layers formed on the membrane surface of the bioreactor and retain microorganisms by incorporating a hybrid growth system (attached and suspended growth). A predetermined volume of sponge cubes can be added into the SMBR reactor to function with the biomass in the reactor to improve biomass growth while also helping to reduce membrane fouling, while cleaning the membrane surface and improving nitrogen and phosphorous removal. The mixture of the present invention can be used with the sponge in an SMBR to operate concurrently in further increasing the biomass, reducing membrane fouling and increasing nutrient removal.

Conventional flocculation is a process that is widely used in water treatment wherein fine particulates in the water are caused to clump together into floc. This floc may then float to the top of the water stream or settle to the bottom and can then be readily removed from the water. Flocculants, or flocculating agents, are chemicals that promote flocculation by causing colloids and other suspended particles in the water to aggregate, forming a floc. Flocculants are generally used in water treatment processes to improve the sedimentation of small waste particles in the water. Particles finer than a micron in size remain continuously in motion in the water due to electrostatic charge (often negative) which causes them to repel each other. The coagulant chemical neutralises this electrostatic charge, and the finer particles begin to collide and agglomerate. These agglomerated heavier particles are called flocs. Many flocculants are multivalent cations such as aluminium, iron, calcium or magnesium. Long chain polymer flocculants, such as modified polyacrylamides, are also commonly used in water treatment processes. These are positively charged molecules which interact with the negatively charged particles reduce the barriers to aggregation. In addition, these chemicals may react with the water to form insoluble hydroxides, under appropriate pH and temperature conditions, which link together to form long chains, physically trapping smaller particles into the floc.

The mixture of the present invention, however, uses a flocculant as part of a biological enhancement technique as opposed to the conventional chemical process described above. While a conventional flocculant or flocculating agent functions as a chemical that neutralises the electrostatic charge on the finer particles in water to allow them to collide and agglomerate, the mixture of the present invention is designed to promote the activity of the existing biological process by providing more nutrients to the biomass to increase their bioactivity. This increased bioactivity has been shown to lead to an increase in the size of the floc, and further increased bioactivity thereby enhancing the existing biological water treatment process. While the process of the present invention is not entirely understood, at least in some part it is believed that the increased and maintained bioactivity in the floc results from the micronutrients in the mixture being brought to the floc during the flocculation process. The biomass in the floc is then readily maintained while simultaneously increasing the size of the floc.

The mixture/reagent of the present invention provides a number of significant and simultaneous advantages over the prior art by enhanced biological treatment of water. The flocculant/micronutrient/polymer mixture enhances the size of the floc, increases bioactivity of the floc and reduces fouling while providing improved treatment. As will be clear to a person skilled in the art, since the flocs are larger not only will there be greater biomass within the floc, more nutrients are provided to the biomass to sustain bioactivity over a longer period of time. This simultaneous synergistic effect of improving bioactivity and reducing fouling by means of the bigger flocs, provides considerable benefit as compared with conventional techniques.

Most conventional techniques have simply applied relevant reagents to provide a desired outcome. In some cases, multiple reagents have a negative effect on the desired outcome. The present invention, on the other hand, provides an enhancement of the biological water treatment by, amongst other things, modifying the characteristics of the floc in a desired way, eg increasing size, improved bioactivity. Such enhanced treatment and modification of the floc characteristics is absent from conventional treatment.

### EXAMPLE 1

### Anti-Fouling Properties of Biodegradable Water Treatment Mixture in membrane Bioreactor (MBR)

Studies of MBR treatment systems have demonstrated that addition of the flocculant/micronutrient/polymer mixture can result in a system trans-membrane pressure (TMP) which is maintained at a low level, only requiring back flushing every 12 hours. This is compared with every 1-2 hours for some MBR plants.

In a preferred embodiment, a natural, starch-based flocculant is included in the mixture to enhance the performance of the MBR, whilst remaining biodegradable, unlike some non-organic or synthetic products, which carry secondary environmental concerns. The significant reduction in membrane fouling which is achieved not only reduces the need to back-flush or clean the membranes, but also increases the critical flux characteristics of the system, which in turn increases the overall capacity of the MBR.

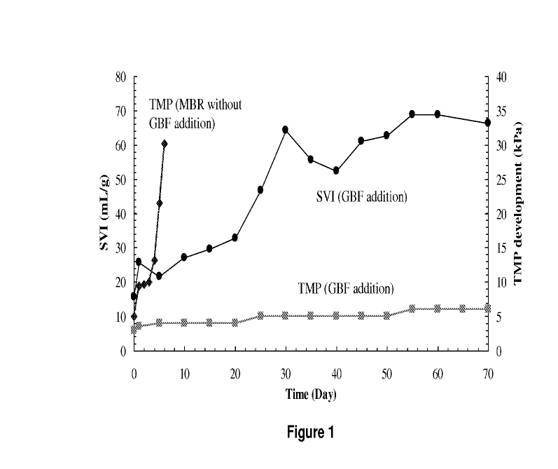

FIG. 1 shows how the addition of a preferred embodiment of the invention (labelled here as GBF) can maintain the Trans-Membrane Pressure at below 6 kPa, and maintain the Sludge Volume Index, an indication of effective sludge settlement, at a healthy level.

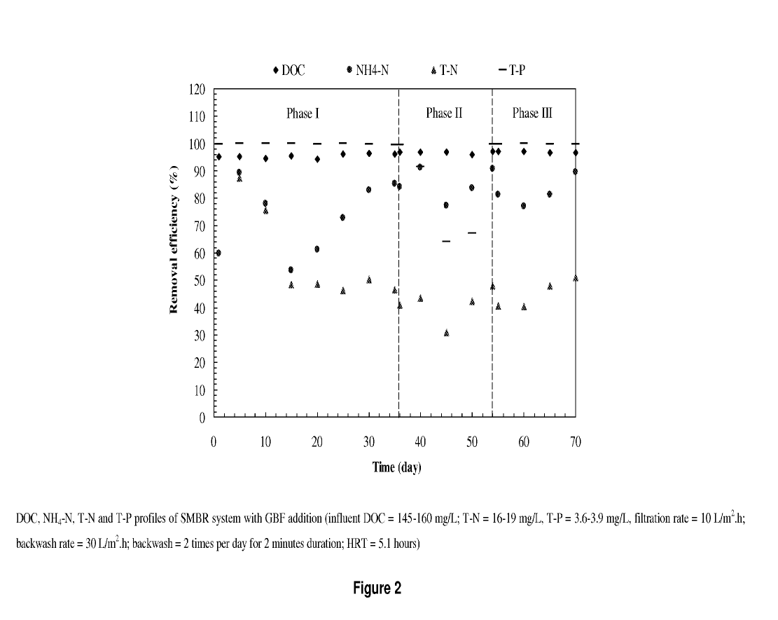

FIG. 2 shows the very high organic removal and near total phosphorous removal in an MBR using a preferred embodiment of the invention. These results compete favourably with alternative flocculant products on the market, but with the advantage of very low membrane fouling.

The ratios of water treatment mixture components used in the analysis presented in FIGS. 1 and 2 are as follows:

Starch-based flocculant: 1000±100

Polymer Nutrient (Chitosan): 25±10

Polymer Nutrient (FeCl₃): 80±20

Polymer Nutrient (MgSO₄): 50±10

Polymer Nutrient (Na₂SO₄): 50±10

Polymer Nutrient (ZnSO₄): 1 to 5

The daily dose of water treatment mixture in Examples 1 and 2 ranged from 10 mg/L to 20 mg/L of treated water with the treatment of synthetic domestic waste.

For industrial water or other water, the dose will be varied according to COD and BOD levels.

### EXAMPLE 2

### Preparation of a Biodegradable Water Treatment Mixture (GBF)

In this embodiment (GBF) the flocculant/micronutrient/polymer mixture is in liquid form and can be simply handled. It can be dosed to the biological treatment system, ie membrane bioreactor, directly once per day. The procedure for making the mixture is as follows:
1. Prepare micronutrient component as 1% solutions of ferric chloride (FeCl₃), magnesium sulphate (MgSO₄), sodium sulphate (Na₂SO₄) and zinc sulphate (ZnSO₄);
2. Prepare polymer component as a chitosan solution using 1% acetic acid;
3. Disperse flocculant as 1000 mg cationic starch (CS) in distilled water to make 50 mL solution;
4. Mix the components using the CS solution at the

CS:chitosan:FeCl₃:MgSO₄:Na₂SO₄:ZnSO₄ ratio of 1000:25:80:50:50:1.

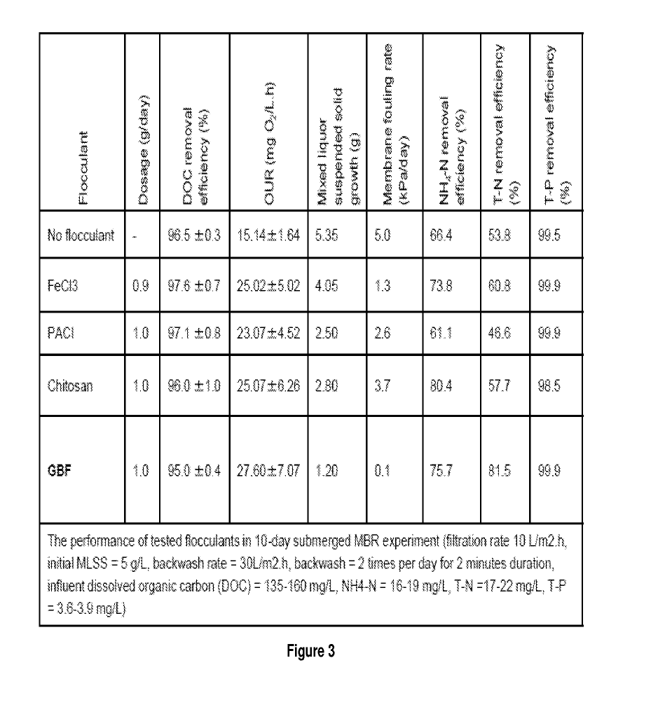

FIG. 3 shows a comparison of four different kinds of flocculants with the inventive mixture (GBF), including two metal salt flocculants (FeCl₃and PACl) and one naturally-occurring polymer (Chitosan). These results were based on 10 days submerged MBR experiments. The data presented in FIG. 3 highlights the synergistic effect of the mixture of the present invention, contributing to enhanced bioactivity and increased phosphorus and nitrogen removal. The embodiment of the invention also demonstrates significantly improved anti-fouling properties, whilst competing favourably against other key criteria.

### EXAMPLE 3

### Study of Biodegradable Water Treatment Mixture in Submerged Membrane Bioreactor (SMBR)

A biodegradable water treatment mixture according to the invention (GBF) was produced using a natural starch-based cationic flocculant (HYDRA Ltd., Hungary). As discussed above, the mixture according to the present invention offers significant advantages over inorganic and synthetic polymer flocculants such as being derived from a renewable source of raw materials, very low cost, and readily degradable in the environment after use. In SMBR, microorganisms also can utilize the carbon source from flocculated bioflocs for microbial activity. The trial dose of the mixture in this study was 1000 mg/day at the first 10 days and 500 l mg/day afterwards.

Submerged Membrane Bioreactor (SMBR) Set-Up A polyethylene hollow fiber membrane module was used with the pore size of 0.1 lm and surface area of 0.195 m2 (Mitsubishi-Rayon, Japan). The effective volume of the bioreactor was 10 L and the permeate flux was maintain at 10 L/m2 h. To save energy in the SMBR, the SMBR, filtrate backwash was conducted only 2 times/day for 2-min duration at a backwash rate of 30 L/m2 h. A pressure gauge was used to measure the trans-membrane pressure (TMP) and a soaker hose air diffuser was used to maintain the air flow rate. The SMBR was filled with sludge from local Wastewater Treatment Plant and acclimatized to synthetic wastewater. The initial mixed liquor suspended solids (MLSS) and biomass mixed liquor volatile suspended solids (MLVSS) concentration were 5 and 4.4 g/L, respectively.

Results and Discussion

Organic and Nutrient Removals

The operation of the SMBR was divided into three phases:
Phase I (biomass growth phase);
Phase II (phosphorus removal recovery phase); and
Phase III (steady phase).

The results of DOC, NH4—N, T-P and T-P removals are shown in FIG. 2.

During Phase I (1-36 days run), the SMBR was operated with complete sludge retention and low initial active microorganism concentration. The biomass mass increased gradually from 4.4 to 14.2 g/L with high DOC and T-P removal efficiency (>95% and >99.5%, respectively) which means the inventive flocculant/micronutrient/polymer mixture could enhance the biological phosphorus removal by biomass metabolism. However, as the cell growth associated mass balance of phosphorus decreased from 0.81 to 0.27 mg P/g biomass synthesis, the phosphorus removal broke down after 36-day run.

In Phase II (37-54 days run), the system had the highest MLVSS concentration of 15.4 g/L on the 40th day, but only 91.4% of T-P was eliminated. Therefore, sludge was withdrawn from the system for next 13 days (up to 53rd day) and the MLSS dropped to 10 g/L. On the 54 th day, 4 g/L(reactor volume) fresh sludge (same acclimatized activated sludge used at the beginning of the experiment) was added into the reactor, which gained the MLSS of 14 g/L and led to high T-P removal again (99.7%). In spite of changing mixed liquor conditions, the organic removal of the system was not affected and the removal still retained as high as previously.

Starting from Phase III (55-70 days run), sludge is wasting from the system according to the biomass growth, which resulted in a sludge retention time (SRT) of 40 days. The system has been running steadily with consistently high DOC and T-P removal (>96.5% and >99.7%, respectively).

Compared with DOC and T-P removal, the system could not achieve high nitrogen removal. At the first 20 days, the bioreactor was supplied with 10 L/min air. With the biomass growth, nitrification reduced rapidly due to dissolved oxygen (DO) decreasing in suspension. Thus, the aeration rate was adjusted up to 12 L/min (DO=7±0.28 mg/L) from 20th day in order to restore nitrification rate. After 30 days, the nitrification rate could maintain constantly around 20-30 mg NH4—N/L h with ammonia removal of 80-90%. Nevertheless, the system had moderate T-N removal which was kept at 40-50% up to 70-day operation.

Respiration Test and SOUR

Respiration tests were conducted using YSI 5300 Biological Oxygen Monitor for testing the impact of the inventive flocculant/micronutrient/polymer mixture on microbial activity or oxygen transfer. Mixed liquor has been taken from the bioreactor periodically in order to measure DO consumption rate, oxygen uptake rate (OUR) and specific oxygen uptake rate (SOUR). As shown in the table below, without GBF addition, the DO consumption measured at days 0 and 5 was 48% and 58% in accordance with low OURs (15.33 and 18.52 mg O2/L h, respectively) and SOURs (3.52 and 3.43 mg O2/g ML VSS h, respectively).

At the same conditions, but with the addition of the inventive flocculant/micronutrient/polymer mixture, the DO consumption and OUR increased dramatically (>30 mg O2/L h) and could maintain a high consumption level (>97.5%) during the Phase I and Phase III, suggesting that the mixture plays an important role in the increase. On the other hand, the values of SOUR dropped in association with the biomass growth in Phase I and then kept constant (>4.2 mg O2/g ML VSS h) in Phase III.

The experimental data shows that the inventive mixture and methods are supportive to biomass activity and non-biotoxic to biomass, as illustrated in FIGS. 2 and 3.

### EXAMPLE 4

### SVI and Membrane Fouling

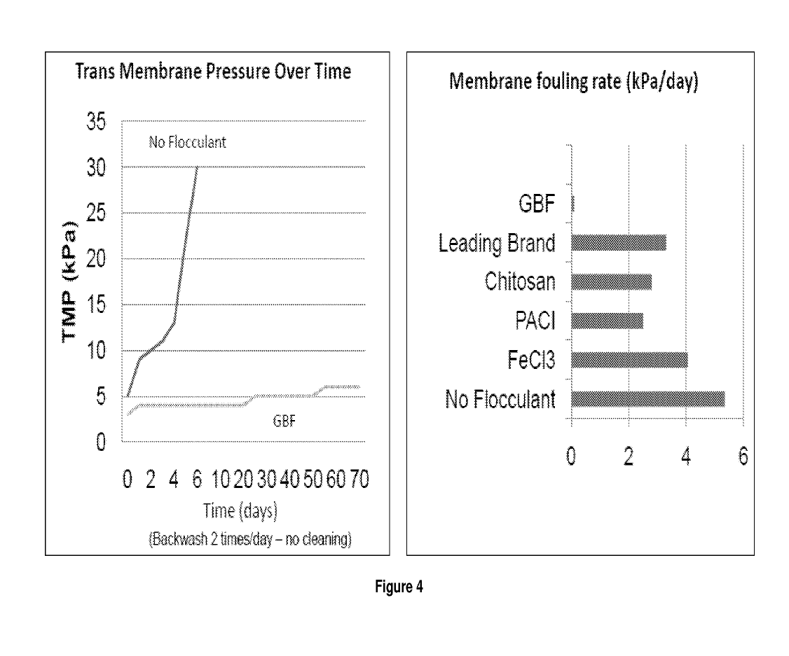

In this study, sludge volume index (SVI) and TMP were investigated as indicators of membrane fouling. A comparison between SMBR with addition of the inventive mixture to an SMBR without applying bioflocculant was carried out at the same operation conditions. Within 6 days operation, the SVI of mixed liquor retained around 50 mL/g and TMP increased up to 30.2 kPa. In contrast, SMBR with the mixture addition resulted in lower SVI (22.6 mL/g) on 6th day, which indicates the predominance of flocs in sludge suspension. In addition, the system exhibited excellent fouling control through TMP development. The TMP of the system only increased from 3.5 to 6 kPa after 70 days operation without any cleaning processes except filtrate backwash 2 times/day with 2-min duration. These results clarified that the inventive mixture (designated as “GBF”) could significantly reduce membrane fouling through modifying the mixed characteristics, as shown in FIG. 4.

The conventional aerated SMBR with low dose mixture addition led to high organic and T-P removals (>95% and >99.5%, respectively). One of the most important advantages of the inventive mixture and its use in biological water treatment systems is seen through its ability to significantly reduce membrane fouling

(TMP development of 2.5 kPa after 70 days of operation) and energy consumption (less backwash frequency). The embodiments show enhanced microbial activity of activated sludge with high DO consumption, high OUR and stable SOUR.

### EXAMPLE 5

### A Water Treatment Mixture Used in and Aerobic Nonwoven Bioreactor

A preferred embodiment of the water treatment mixture of the present invention (designated as GBF) was also tested in nonwoven bioreactors. When the mixture/reagent was employed in an anaerobic nonwoven bioreactor, the denitrification of the system was shown to be significantly improved and more than 96% of nitrate was removed from the wastewater (initial nitrate concentration=17.5-20 mg/L).

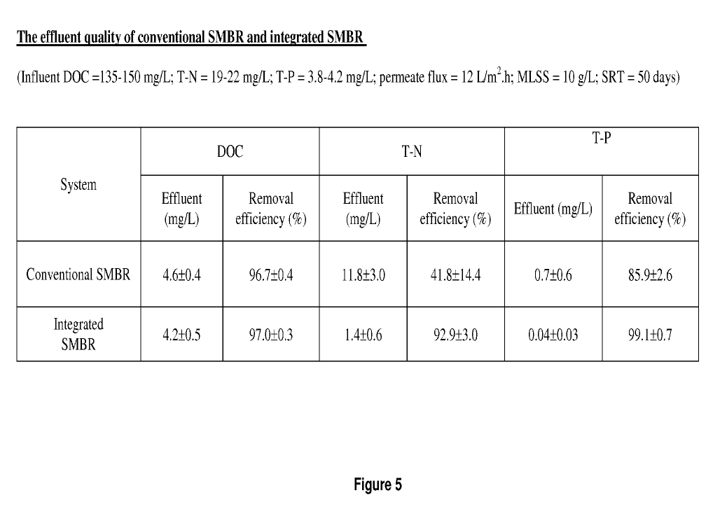

An anoxic nonwoven bioreactor was also tested in combination with a submerged membrane bioreactor. The integrated system had substantially improved performance, with improved total nitrogen removal and reduced membrane fouling, compared to conventional SMBR. The results of this study are presented in FIG. 5.

### INDUSTRIAL APPLICABILITY

It is clear that the use of the mixture, reagent and treatment process according to the present invention provides significant advantages over the prior art. It is particularly suitable but not limited to a biological water treatment system. The disclosed mixtures, reagent and methods according to the present invention improve various aspects of water treatment while minimising damage to the biomass. Reduced fouling of upstream treatment operations also results.

Although the invention has been described with reference to specific examples, it will be appreciated by those skilled in the art that the invention may be embodied in many other forms.

1. A mixture for treating water in a biological water treatment process comprising:
an organic-based flocculant;
a micronutrient; and
a polymer;
wherein said flocculant, said micronutrient and said polymer are mixed in a predetermined ratio to enhance said biological water treatment.

2. A mixture for treating water according to claim 1 wherein said flocculant, said micronutrient and said polymer are added to said water in synergistic quantities so as to enhance said biological water treatment.

3. A mixture for treating water according to claim 1 or claim 2 wherein the mixture comprises 20-60 parts per weight of said flocculant, 4-8 parts per weight of said micronutrient and 1-5 parts per weight of said polymer.

4. A mixture for treating water according to any one of the preceding claims wherein said flocculant is provided in a quantity of between 8 and 17 mg per litre of water, said micronutrient is provided in a quantity of between 0.2 and 1 mg per litre of water and said polymer is provided in a quantity of between 1.5 and 3 mg per litre of water.

5. A mixture for treating water according to any one of the preceding claims wherein said flocculant is biodegradable.

6. A mixture for treating water according to any one of the preceding claims wherein said flocculant is a natural organic-based flocculant.

7. A mixture for treating water according to any one of the preceding claims wherein said flocculant is a starch-based flocculant.

8. A mixture for treating water according to any one of the preceding claims wherein said micronutrient comprises a salt selected from a group consisting of iron, zinc, sodium, magnesium and manganese salts.

9. A mixture for treating water according to claim 8 wherein said micronutrient comprises a plurality of inorganic salts.

10. A mixture for treating water according to claim 8 or claim 9 wherein said micronutrient comprises one or more of ferric chloride (FeCl₃), magnesium sulphate (MgSO₄), sodium sulphate (Na₂SO₄), zinc sulphate (ZnSO₄) and manganese chloride (MnCl₂).

11. A mixture for treating water according to any one of claims 1 to 7 wherein said micronutrient comprises yeast.

12. A mixture for treating water according to any one of the preceding claims wherein said polymer is a naturally occurring polymer.

13. A mixture for treating water according to any one of the preceding claims wherein said polymer is chitosan.

14. A reagent for a biological water treatment process, said reagent comprising:
a micronutrient; and
a naturally occurring polymer;
wherein said reagent is applied to said process in synergistic quantities with a flocculant.

15. A reagent according to claim 14 wherein said reagent is applied to said process prior to or simultaneously with said flocculant.

16. A reagent according to claim 14 or claim 15 wherein the reagent comprises 4-8 parts per weight of said micronutrient and 1-5 parts per weight of said polymer, and is applied with 20-60 parts per weight of said flocculant.

17. A reagent according to any one of claims 14 to 16 wherein the polymer/micronutrient ratio is between 0.2/1.5 mg and 1/3 mg per litre of treated water.

18. A reagent according to any one of claims 14 to 17 wherein said micronutrient comprises a salt selected from a group consisting of iron, zinc, sodium, magnesium and manganese salts.

19. A reagent according to claim 18 wherein said micronutrient comprises a plurality of inorganic salts.

20. A reagent according to claim 18 or claim 19 wherein said micronutrient comprises one or more of ferric chloride (FeCl₃), magnesium sulphate (MgSO₄), sodium sulphate (Na₂SO₄), zinc sulphate (ZnSO₄) and manganese chloride (MnCl₂).

21. A reagent according to any one of claims 14 to 17 wherein said micronutrient comprises yeast.

22. A reagent according to any one of claims 14 to 21 wherein the polymer is a naturally occurring polymer.

23. A reagent according to any one of claims 14 to 22 wherein said polymer is chitosan.

24. A biological water treatment system comprising:
an organic-based flocculant;
a micronutrient; and
a polymer;
said flocculant, said micronutrient and said polymer being mixed in a predetermined synergistic ratio and added to water in a biological water treatment process.

25. A biological water treatment system according to claim 24 wherein said flocculant, micronutrient and polymer are mixed together in said predetermined synergistic ratio prior to addition to the water treatment process.

26. A biological water treatment system according to claim 24 wherein said flocculant, micronutrient and polymer are mixed in situ said water treatment process.

27. A biological water treatment system according to any one of claims 24 to 26 wherein said biological water treatment process includes a membrane bioreactor.

28. A biological water treatment system according to any one of claims 24 to 26 wherein said biological water treatment process includes a submerged sponge.

29. A biological water treatment system according to any one of claims 24 to 26 wherein said biological water treatment process includes a submerged membrane bioreactor.

30. A biological water treatment system according to any one of claims 24 to 29 comprising 20-60 parts per weight of said flocculant, 4-8 parts per weight of said micronutrient and 1-5 parts per weight of said polymer.

31. A biological water treatment system according to any one of claims 24 to 30 wherein said flocculant is provided in a quantity of between 8 and 17 mg per litre of water, the micronutrient is provided in a quantity of between 0.2 and 1 mg per litre of water and the polymer is provided in a quantity of between 1.5 and 3 mg per litre of water.

32. A biological water treatment system according to any one of claims 24 to 31 wherein said flocculant is biodegradable.

33. A biological water treatment system according to any one of claims 24 to 32 wherein said flocculant is a natural organic-based flocculant.

34. A biological water treatment system according to claim 33 wherein said flocculant is a starch-based flocculant.

35. A biological water treatment system according to any one of claims 24 to 34 wherein said micronutrient comprises a salt selected from a group consisting of iron, zinc, sodium, magnesium and manganese salts.

36. A biological water treatment system according to claim 35 wherein said micronutrient comprises a plurality of inorganic salts.

37. A biological water treatment system according to claim 35 or claim 36 wherein said micronutrient comprises one or more of ferric chloride (FeCl₃), magnesium sulphate (MgSO₄), sodium sulphate (Na₂SO₄), zinc sulphate (ZnSO₄) and manganese chloride (MnCl₂).

38. A biological water treatment system according to any one of claims 24 to 34 wherein said micronutrient comprises yeast.

39. A biological water treatment system according to any one of claims 24 to 38 wherein said polymer is a naturally occurring polymer.

40. A biological water treatment system according to any one of claims 24 to 39 wherein said polymer is chitosan.

41. A biological method of treating water comprising the steps of:
adding a micronutrient, a polymer and an organic-based flocculant in a predetermined synergistic ratio to water in need of treatment; and
allowing the resultant treated water to form floc.

42. A biological method of treating water according to claim 41 further comprising removing said floc from said water.

43. A biological method of treating water according to claim 41 or claim 42 wherein said micronutrient, said polymer and said organic-based flocculant are added separately to said water.

44. A biological method of treating water according to claim 41 or claim 42 wherein said micronutrient and/or said polymer are added prior to or simultaneously with said flocculant.

45. A biological method of treating water according to claim 41 or claim 42 wherein said micronutrient, said polymer and said organic-based flocculant are mixed together prior to addition to said water.

46. A biological method of treating water according to any one of claims 41 to 45 wherein 20-60 parts per weight of said flocculant, 4-8 parts per weight of said micronutrient and 1-5 parts per weight of said polymer are added to said water.

47. A biological method of treating water according to any one claims 41 to 46 wherein said flocculant is provided in a quantity of between 8 and 17 mg per litre of water, the micronutrient is provided in a quantity of between 0.2 and 1 mg per litre of water and the polymer is provided in a quantity of between 1.5 and 3 mg per litre of water.

48. A biological method of treating water according to any one of claims 41 to 47 wherein said flocculant is biodegradable.

49. A biological method of treating water according to any one of claims 41 to 48 wherein said flocculant is a natural organic-based flocculant.

50. A biological method of treating water according to claim 49 wherein said flocculant is a starch-based flocculant.

51. A biological method of treating water according to any one of claims 41 to 50 wherein said micronutrient comprises a salt selected from a group consisting of iron, zinc, sodium, magnesium and manganese salts.

52. A biological method of treating water according to claim 51 wherein said micronutrient comprises a plurality of inorganic salts.

53. A biological method of treating water according to claim 51 or claim 52 wherein said micronutrient comprises one or more of ferric chloride (FeCl₃), magnesium sulphate (MgSO₄), sodium sulphate (Na₂SO₄), zinc sulphate (ZnSO₄) and manganese chloride (MnCl₂).

54. A biological method of treating water according to any one of claims 41 to 50 wherein said micronutrient comprises yeast.

55. A biological method of treating water according to any one of claims 41 to 54 wherein said polymer is a naturally occurring polymer.

56. A biological method of treating water according to claim 55 wherein said polymer is chitosan.

57. A method of enhancing the efficacy of an organic-based flocculant in the biological treatment of water, said method comprising combining said flocculant with a synergistic quantity of a micronutrient and polymer either prior to or simultaneously with addition of the flocculant to the water.

58. A method as claimed in claim 57 wherein combining said micronutrient and polymer with said organic based flocculant is conducted within a biological water treatment apparatus for treating said water.

59. A method of modifying characteristics of floc produced by using a flocculant in a biological water treatment process comprising:
adding to water in need of treatment, either separately to or simultaneously with said flocculant, a micronutrient and a polymer in a predetermined synergistic ratio.

60. A method of modifying characteristics of floc according to claim 59 wherein said characteristic is one or more of size of said floc, biological activity, density, settling rate, viscosity, surface properties, sludge volume index (SVI) and zone settling velocity (ZSV).

61. A method of modifying characteristics of floc claim 59 or claim 60 wherein 20-60 parts per weight of said flocculant, 4-8 parts per weight of said micronutrient and 1-5 parts per weight of said polymer are added to said water.

62. A method of modifying characteristics of floc according to any one claims 59 to 61 wherein said flocculant is provided in a quantity of between 8 and 17 mg per litre of treated water, the micronutrient is provided in a quantity of between 0.2 and 1 mg per litre of treated water and the polymer is provided in a quantity of between 1.5 and 3 mg per litre of treated water.

63. A method of modifying characteristics of floc according to any one of claims 59 to 62 wherein said flocculant is biodegradable.

64. A method of modifying characteristics of floc according to any one of claims 59 to 63 wherein said flocculant is a natural organic-based flocculant.

65. A method of modifying characteristics of floc according to claim 64 wherein said flocculant is a starch-based flocculant.

66. A method of modifying characteristics of floc according to any one of claims 59 to 65 wherein said micronutrient comprises a salt selected from a group consisting of iron, zinc, sodium, magnesium and manganese salts.

67. A method of modifying characteristics of floc according to claim 66 wherein said micronutrient comprises a plurality of inorganic salts.

68. A method of modifying characteristics of floc according to claim 66 or claim 67 wherein said micronutrient comprises one or more of ferric chloride (FeCl₃), magnesium sulphate (MgSO₄), sodium sulphate (Na₂SO₄), zinc sulphate (ZnSO₄) and manganese chloride (MnCl₂).

69. A method of modifying characteristics of floc according to any one of claims 59 to 65 wherein said micronutrient comprises yeast.

70. A method of modifying characteristics of floc according to any one of claims 59 to 69 wherein said polymer is a naturally occurring polymer.

71. A method of modifying characteristics of floc according to claim 70 wherein said polymer is chitosan.

72. A method of treating water comprising the steps of:
adding to said water an organic-based flocculant, a micronutrient, and a polymer;
wherein said flocculant, said micronutrient and said polymer are mixed into said water in a predetermined synergistic ratio to enhance flocculation of particles in said water.

73. A method of treating water comprising the steps of:
adding to said water an organic-based flocculant, a micronutrient, and a polymer;
wherein said flocculant, said micronutrient and said polymer are mixed into said water in a predetermined synergistic ratio to reduce fouling on a surface of a membrane in a membrane bioreactor used to treat said water.

74. A method of treating water comprising the steps of:
adding to said water an organic-based flocculant, a micronutrient, and a polymer;
wherein said flocculant, said micronutrient and said polymer are mixed into said water in a predetermined synergistic ratio to improve an uptake of phosphorous and/or nitrogen by a biomass in a biological treatment system used to treat said water.

75. A method of treating water comprising the steps of:
adding to said water an organic-based flocculant, a micronutrient, and a polymer;
wherein said flocculant, said micronutrient and said polymer are mixed into said water in a predetermined synergistic ratio to improve bioactivity of a biomass and/or floc in a biological treatment system used to treat said water.

76. A method of treating water comprising the steps of:
adding to said water an organic-based flocculant, a micronutrient; and a polymer;
wherein said flocculant, said micronutrient and said polymer are mixed into said water in a predetermined synergistic ratio to improve flux in a membrane bioreactor used to treat said water.
